(19) United States
(12) Patent Application Publication
(10) Pub. No.: US 2026/0114907 A1
(43) Pub. Date: Apr. 30, 2026

(54) INTERSPINOUS SPACER WITH RELATIVELY FEW COMPONENTS AND METHODS AND SYSTEMS

(71) Applicant: Boston Scientific Neuromodulation Corporation, Valencia, CA (US)

(72) Inventor: Thomas Paul Lopez, Sunland, CA (US)

(21) Appl. No.: 19/371,683

(22) Filed: Oct. 28, 2025

Related U.S. Application Data
(60) Provisional application No. 63/713,518, filed on Oct. 29, 2024.

(51) Int. Cl. A61B 17/70; A61B 17/00; A61B 17/56

(52) U.S. Cl. CPC A61B 17/7065; A61B 17/7064; A61B 2017/00477; A61B 2017/564

(57) ABSTRACT

An interspinous spacer has a first arm and a second arm each including a body and two receiving extensions extending distally from the body, the body including a bridge extending between the two receiving extensions and two walls extending proximally from the bridge. The first arm includes a latch extending distally from a proximal end of one of the walls and the second arm includes a lip extending from a proximal end of one of the walls. The first and second arms are coupled together by interleaving the two walls of the first and second arms and, when coupled together, the first and second arms are rotatable from an implantation position with the receiving extensions of the first and second arms extending in a same direction to a deployed position with the receiving extensions of two arms extending in opposite directions.

### CROSS-REFERENCE TO RELATED APPLICATIONS

This application claims the benefit under 35 U.S.C. § 119(e) of U.S. Provisional Patent Application Ser. No. 63/713,518, filed Oct. 29, 2024, which is incorporated herein by reference.

### FIELD

The present invention is directed to the area of interspinous spacers for deployment between adjacent spinous processes. The present invention is also directed to interspinous spacers with three or fewer parts, as well as systems and methods for utilizing the interspinous spacers.

### BACKGROUND

With spinal stenosis, the spinal canal narrows and pinches the spinal cord and nerves, causing pain in the back and legs. Typically, with age, a person's ligaments may thicken, intervertebral discs may deteriorate, or facet joints may break down. The conditions can contribute to the narrowing of the spinal canal Injury, heredity, arthritis, changes in blood flow, and other causes may also contribute to spinal stenosis.

Various treatments of the spine have been proposed or used including medications, surgical techniques, and implantable devices that alleviate and reduce pain associated with the back. In one surgical technique, a spacer is implanted between adjacent spinous processes of a patient's spine. The implanted spacer opens the spinal canal, maintains the desired distance between vertebral body segments, and, as a result, avoids or reduces impingement of nerves and relieves pain. For suitable candidates, an implantable interspinous spacer may provide significant benefits in terms of pain relief.

### BRIEF SUMMARY

One aspect is an interspinous spacer having a first arm including a body and two receiving extensions extending distally from the body, the body including a bridge extending between the two receiving extensions, two walls extending proximally from the bridge, and a latch extending distally from a proximal end of a one of the two walls of the body of the first arm and extending between the two walls of the body of the first arm; and a second arm including a body and two receiving extensions extending distally from the body, the body including a bridge extending between the two receiving extensions, two walls extending proximally from the bridge, and a lip extending from a proximal end of a one of the two walls of the body of the second arm and extending between the two walls of the body of the second arm; wherein the first arm and the second arm are coupled together by interleaving the two walls of the first and second arms, wherein the first arm and second arm, when coupled together, are rotatable from an implantation position with the two receiving extensions of both the first arm and second arm extending in a same direction to a deployed position with the two receiving extensions of the first arm extending in an opposite direction from the two receiving extensions of the second arm, wherein, in the deployed position, the latch of the body of the first arm is configured to engage the lip of the body of the second arm when the first arm and the second arm are pulled away from each other to prevent or hinder decoupling of the first arm from the second arm.

In at least some aspects, the interspinous spacer consists of the first arm and the second arm.

In at least some aspects, each of the first arm and the second arm includes a post extending from a one of the two walls and an opening in another one of the two walls, wherein the post of each of the first arm and the second arm is configured to extend into the opening of the other one of the first arm and the second arm.

In at least some aspects, a one of the first arm or the second arm includes a receiving appendage extending from the body and another one of the first arm or the second arm includes a post configured to be received within the receiving appendage.

In at least some aspects, the interspinous spacer further includes a post, wherein each of the first arm and the second arm includes an opening in at least one of the two walls for receiving the post extending through each of the openings of the first arm and the second arm.

In at least some aspects, a one of the two walls of each of the first arm and the second arm includes an undercut attachment portion for attachment of a clamp of a spacer insertion instrument. In at least some aspects, the body of each of the first arm and the second arm includes a cam surface for engagement of a cam actuator of a spacer insertion instrument to facilitate rotation of the first arm and the second arm from the implantation position to the deployed position.

In at least some aspects, the latch extends along only a first portion of a depth of the one of the two walls of the body of the first arm. In at least some aspects, along at least part of a remaining portion of the depth of the one of the two walls of the body of the first arm, the one of the two walls of the body of the first arm extends laterally toward another one of the two walls. In at least some aspects, along at least part of a remaining portion of the depth of the one of the two walls of the body of the first arm, a portion of a one of the two walls of the body of the second arm extends laterally toward the one of the two walls of the body of the first arm and is configured for positioning adjacent to the latch when the interspinous spacer is in the deployed position.

Another aspect is a kit that includes any of the interspinous spacers described above and a spacer insertion instrument configured to releasably grip the interspinous spacer for implantation into a patient.

In at least some aspects, the spacer insertion instrument includes at least two clamps configured for releasably gripping the interspinous spacer between the clamps and at least one cam actuator configured to engage the first and second arms and, when operated, to rotate the first and second arms from the implantation position to the deployed position. In at least some aspects, each of the at least two clamps includes a clamp extension configured for positioning in an opening in the body of one of the first arm or the second arm for the interspinous spacer for releasably gripping the interspinous spacer. In at least some aspects, the spacer insertion instrument includes a handle portion that, when operated, spreads the at least two clamps apart for loading or unloading of the interspinous spacer from the spacer insertion instrument.

In at least some aspects, the spacer insertion instrument further includes a wedge to engage the latch when the interspinous spacer is in the deployed position and to move the latch so that the first and second arms of the interspinous spacer can be rotated from the deployed position toward the implantation position.

A further aspect is a method for implanting any of the interspinous spacers described above. The method includes inserting into a patient the interspinous spacer in the implantation position and coupled to a spacer insertion instrument; rotating the first and second arms of the interspinous spacer from the implantation position to the deployed position using the spacer insertion instrument; after the rotating to the deployed position, releasing the interspinous spacer from the spacer insertion instrument; and removing the spacer insertion instrument from the patient leaving the interspinous spacer implanted in the patient.

In at least some aspects, the method further includes coupling the interspinous spacer to the spacer insertion instrument using at least two clamps of the spacer insertion instrument. In at least some aspects, the rotating includes operating a cam actuator of the spacer insertion instrument to push against portions of the first and second arms resulting in the rotation of the first and second arms of the interspinous spacer from the implantation position to the deployed position. In at least some aspects, the rotating includes disengaging the latch from engagement with the lip allowing the latch to extend into a cavity between defined the by the walls of the first and second arms.

Yet another aspect is a method for explanting any of the interspinous spacers described above. The method includes coupling a spacer insertion instrument to the interspinous spacer; operating a wedge of the spacer insertion instrument to engage the latch and move the latch toward the one of the walls of the body of the first arm; and causing the first arm and the second arm to move from the deployed position toward the implantation position.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic perspective view of one embodiment of an interspinous spacer in a deployed position;

FIG. 1B is a schematic perspective view of the interspinous spacer of FIG. 1A with the first and second arms separated;

FIG. 1C is a schematic top view of the interspinous spacer of FIG. 1A;

FIG. 1D is a schematic close-up top view of a center portion of the interspinous spacer of FIG. 1A;

FIG. 1E is a schematic top view of the interspinous spacer of FIG. 1A with the first and second arms separated;

FIG. 2A is a schematic perspective view of one embodiment of a spacer insertion instrument with an interspinous spacer attached;

FIG. 2B is a schematic close-up perspective view of the interspinous spacer of FIG. 1A in an implantation position and a distal portion of the spacer insertion instrument of FIG. 2A;

FIG. 2C is a schematic close-up perspective view of the interspinous spacer of FIG. 1A in the deployed position and a distal portion of the spacer insertion instrument of FIG. 2A;

FIG. 2D is a schematic close-up cross-sectional view of a portion of the interspinous spacer of FIG. 1A in the implantation position and a distal portion of the spacer insertion instrument of FIG. 2A;

FIG. 2E is a schematic close-up cross-sectional view of a portion of the interspinous spacer of FIG. 1A in the deployed position and a distal portion of the spacer insertion instrument of FIG. 2A;

FIG. 2F is a schematic close-up perspective view of the interspinous spacer of FIG. 1A in an intermediate position and a distal portion of the spacer insertion instrument of FIG. 2A;

FIG. 2G is a schematic close-up cross-sectional view of a distal portion of the spacer insertion instrument of FIG. 2A;

FIG. 3A is a schematic perspective view of another embodiment of an interspinous spacer in a deployed position with the first and second arms separated;

FIG. 3B is a schematic top view of the interspinous spacer of FIG. 3A with the first and second arms separated;

FIG. 4A is a schematic perspective view of a third embodiment of an interspinous spacer in a deployed position with the first and second arms separated;

FIG. 4B is a schematic top view of the interspinous spacer of FIG. 4A with the first and second arms separated;

FIG. 5A is a schematic perspective view of a fourth embodiment of an interspinous spacer in a deployed position with the first and second arms separated; and

FIG. 5B is a schematic top view of the interspinous spacer of FIG. 3A with the first and second arms separated.

### DETAILED DESCRIPTION

The present invention is directed to the area of interspinous spacers for deployment between adjacent spinous processes. The present invention is also directed to interspinous spacers with three or fewer parts, as well as systems and methods for utilizing the interspinous spacers.

Examples of interspinous spacers are found in U.S. Pat. Nos. 8,123,782; 8,128,662; 8,273,108; 8,277,488; 8,292,922; 8,425,559; 8,613,747; 8,864,828; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference. Unless indicated otherwise, the features and methods described in these references can be applied to the interspinous spacers described herein.

An interspinous spacer is disposed between two adjacent vertebrae, for example, between the spinous processes of two adjacent vertebrae. Conventional interspinous spacers often have at least five separate parts that are assembled to form the spacer. These separate parts are relatively small and can be challenging to manufacture and assemble. In addition, the parts of the interspinous spacer must withstand relatively high loads.

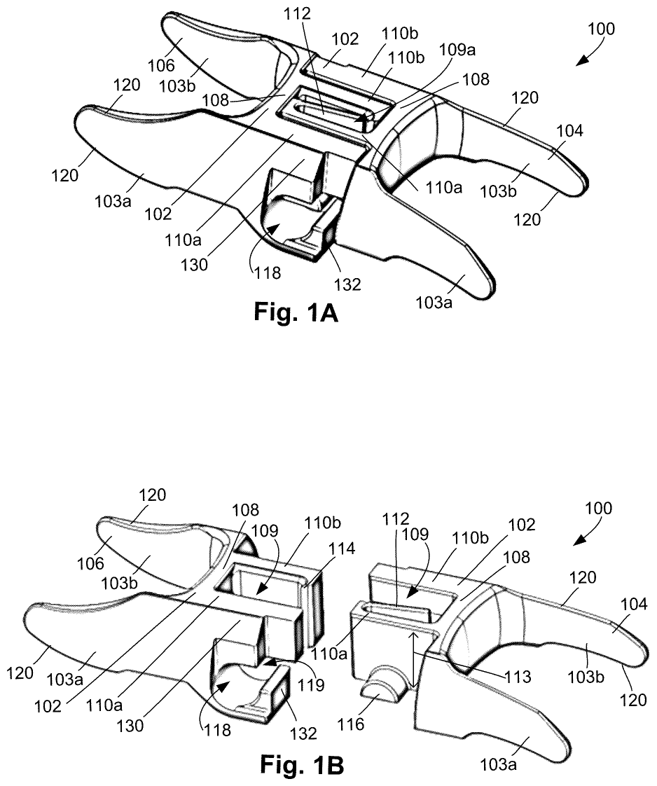

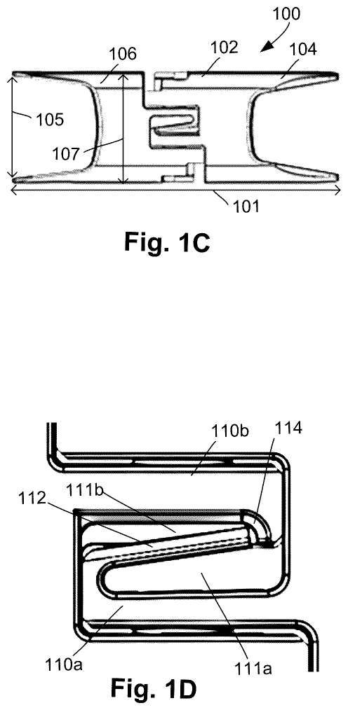

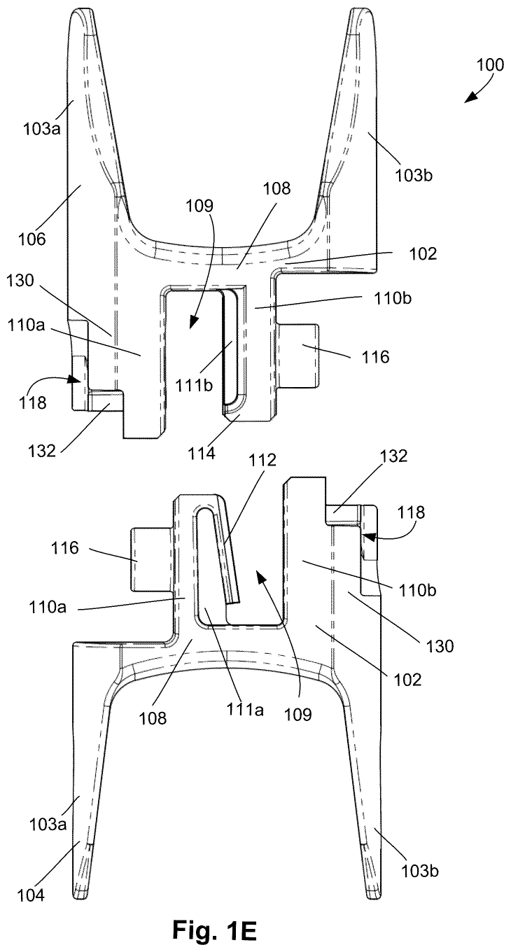

As described herein, in at least some embodiments, an interspinous spacer can be made with three or fewer separate parts. FIGS. 1A to 1E illustrate one embodiment of an interspinous spacer 100 that includes a first (or superior) arm 104 and a second (or inferior) arm 106. In at least some embodiments, the interspinous spacer 100 only includes the first arm 104 and the second arm 106. The first and second arms 104, 106 each include a body 102 and two receiving extensions 103a, 103b extending distally from the body and defining a space between the receiving extensions of each arm for receiving a portion of a vertebrae (e.g., a portion of a spinous process) therebetween. Each body 102 includes a bridge 108 extending between the two receiving extensions 103a, 103b. In at least some embodiments, a face or edge 120 (e.g., an anterior or posterior face or edge) of each receiving extension 103a, 103b can include a concave, convex, or curved portion for conforming to bony anatomy of the vertebra (e.g., the spinous process or lamina of the vertebra) that is received between the receiving extensions.

In at least some embodiments, the length 101 of interspinous spacer is in a range of 7 to 20 mm or a range of 8 to 16 mm, although other lengths can be used. In at least some embodiments, a tip-to-tip distance 105 of the extensions 103a, 103b is in a range of 8 to 12 millimeters, although other distances can be used. In at least some embodiments, a width 107 of the bridge 108 is in a range of 6 to 10 millimeters, although other widths can be used. In at least some embodiments, the tip-to-tip distances 105 or widths 107 or both can be the same for both the first and second arms 104, 106 or can differ between the first arm 104 and the second arm 106. In at least some embodiments, interspinous spacers of different sizes can be made so that a clinician can select which size is to be used for a particular surgery based on, for example, the size and arrangement of the vertebrae.

In at least some embodiments, the first arm 104 (or second arm 106) forms a larger space for receiving the superior vertebra (for example, the superior spinous process) than the space formed by the second arm 106 (or first arm 104) for receiving the inferior vertebra (for example, the inferior spinous process) as vertebrae and spinous processes are naturally narrower on top and wider on the bottom. In at least some embodiments, where there is a difference in size between the first and second arms 104, 106, the spacer 100 may include a marking or other indication so that a clinician can individually identify the first and second arms 104, 106 for correct implantation orientation within the patient. In at least some embodiments, spacers 100 can include a marking or other indication (e.g., color) to distinguish spacers of different sizes (e.g., different lengths, different tip-to-tip distances, or the like or any combination thereof) for use with different vertebrae or differently sized individuals.

In at least some embodiments, the arms 104, 106, as well as any other spacer components, can be made of metal, such as, for example, titanium, titanium alloys, stainless steel, or the like, and are preferably made of a biocompatible metal. In at least some of these embodiments, the interspinous spacer 100 in the deployed position provides metal-to-metal interactions as the spine moves or produces compression, as well as during torsion of the spacer.

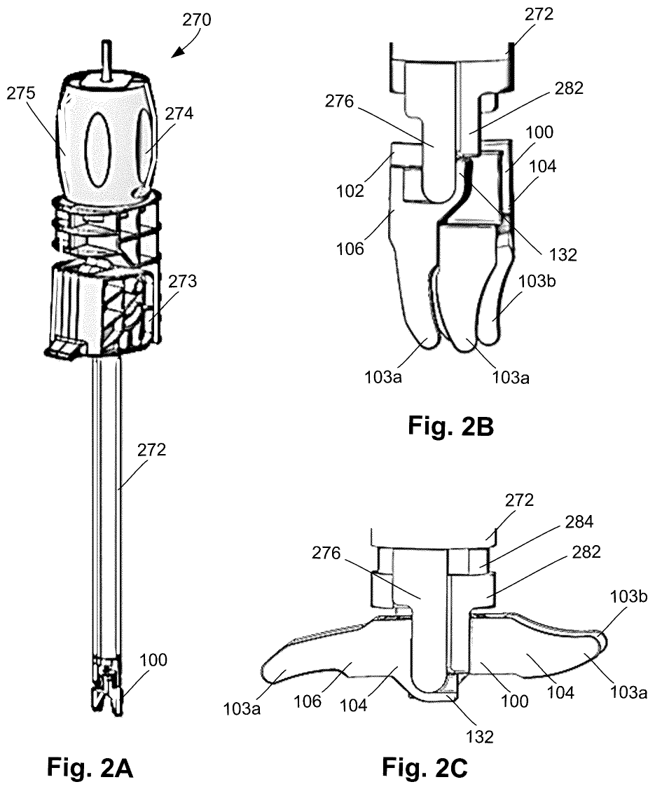

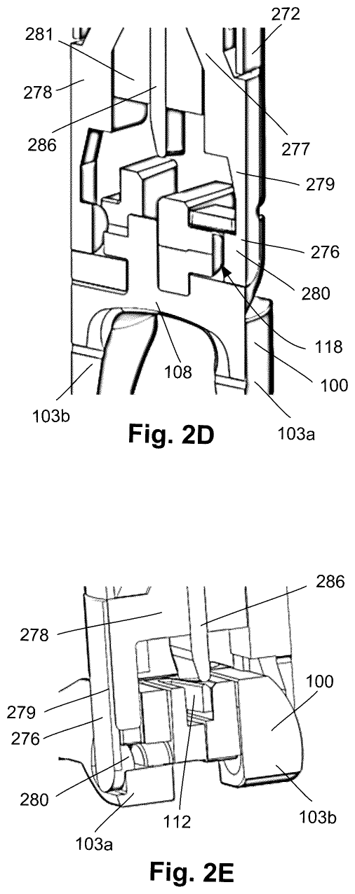

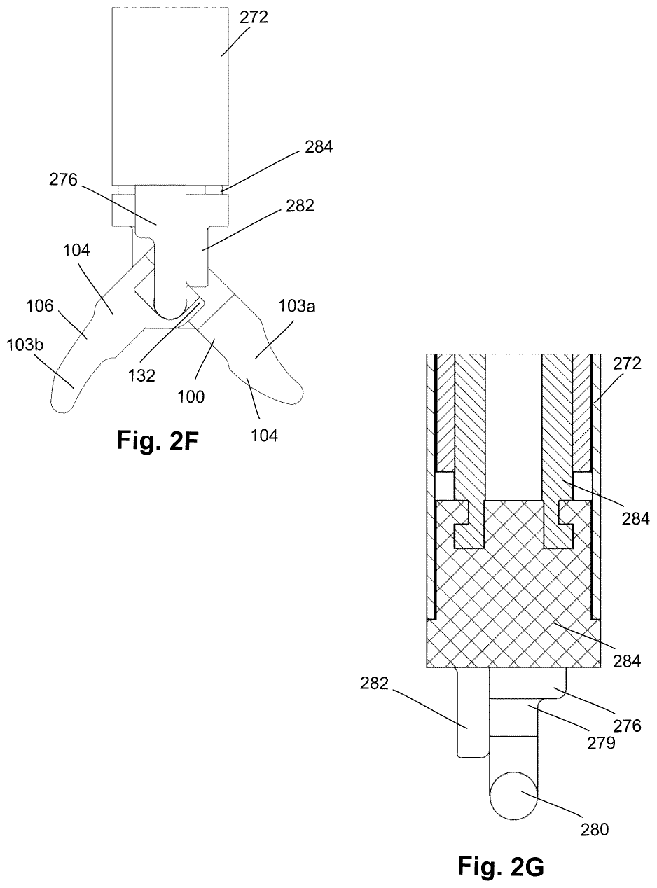

FIGS. 1A, 1C, and 1D illustrate the interspinous spacer 100 in a deployed position with the receiving extensions 103a, 103b of the first arm 104 extending opposite the receiving extensions 103a, 103b of the second arm 104. FIG. 2B illustrates the interspinous spacer 100 in an implantation position with the receiving extensions 103a 103b of the first and second arms 104 extending in the same direction In FIGS. 2B, the interspinous spacer is also attached to a spacer insertion instrument 270, which is further described below. FIG. 2C illustrates the interspinous spacer 100 attached to the spacer insertion instrument 270 and in the deployed position.

The body 102 of each of the first and second arms 104, 106 further includes two walls 110a, 110b extending proximally away from the bridge 108 opposite the two receiving extensions 103a, 103b and defining a cavity 109 therebetween. When the first and second arms 104, 106 are brought together, the walls 110a, 110b of the first and second arms interleave with each other, as illustrated in FIGS. 1A, 1C, and 1D, and define a cavity 109a. In at least some embodiments, assembly of the interspinous spacer 100 includes snapping together the first and second arms 104, 106 in, for example, the implantation position (or, alternatively, the deployed position).

The first arm 104 includes a latch 112 extending distally from a proximal end of one wall 110a and between the two walls 110a, 110b of the first arm, as illustrated in FIGS. 1A to 1D. The second arm 106 includes a lip 114 extending from the proximal end of one wall 110b and between the two walls 110a, 110b of the second arm. In at least some embodiments, in the implantation position, the latch 112 engages the lip 114 along a substantial (or entire) length of the latch and lip. In at least some embodiments, when the first and second arms 104, 106 are rotated from the implantation position to the deployed position, the latch 112 is released from engagement with the lip 114 and the latch expands into the cavity 109a. (It will be understood that the first and second arms 104, 106 could also be pushed together in the implantation position with the latch 112 flexing toward the wall 110a of the first arm 104 (from which the latch extends) to pass the lip 114 and then expanding into the cavity 109a.)

In at least some embodiments, the latch 112 and lip 114, when the interspinous spacer 100 is in the deployed position, maintains the interspinous spacer 100 in the deployed position. In at least some embodiments, application of a force to pull the first and second arms 104, 106 apart, application of a torque, or application of force to rotate the first and second arms toward the implantation position is resisted by the latch 112 engaging the lip 114 to prevent or hinder disengagement of the first arm 104 from the second arm 106 or rotation of the first and second arms away from the deployed position.

In at least some embodiments, the latch 112 extends along an entire depth 113 (or at least 70% of the depth) of the wall 110a. In other embodiments, including the illustrated embodiment of FIGS. 1A to 1E, the latch 112 extends along no more than 70%, 60%, 50%, 40%, 33%, 30%, or 25% of the depth 113 (FIG. 1B) of the wall 110a. In at least some of these embodiments, a portion 111a of the wall 110a of the first arm 104 extends laterally toward the wall 110b of the second arm 104 along at least a portion of the remaining depth of the wall 110a, as illustrated in FIGS. 1D and E. In at least some embodiments, a portion 111b of the wall 110b of the second arm 106 is arranged to extend laterally towards portion 111a when the first and second arms 104, 106 are coupled.

In at least some embodiments, one or both of the first and second arms 104, 106 includes a post 116 with a corresponding opening 118 in the other arm for receiving the post, as illustrated in FIGS. 1A, 1B, and 1E. In at least some embodiments, one or both of the first and second arms 104, 106 includes an entry opening 119 for inserting the post 116 of one arm into the opening 118 of the other arm. In at last some embodiments, the post 116 is a cylinder, truncated cylinder (for example, a half cylinder or “D” post, as illustrated in FIG. 1A), or any other suitably shaped post, axle, or shaft. In at least some embodiments, to facilitate rotation of the post 116 within the opening 118 as the first and second arms are rotated between the implantation position and the deployed position, the post 116 of each arm (or at least the curved portion of the post) and the corresponding opening 118 of the other arm have a curvature or radius of curvature that is the same or differs by no more than 25%, 10%, or 5%.

In at least some embodiments, a method for implanting an interspinous spacer includes making a small midline or lateral-to-midline incision in the patient for percutaneous delivery of the spacer 100. In at least some embodiments, the supraspinous ligament is avoided. In at least some embodiments, the supraspinous ligament is split longitudinally along the direction of the tissue fibers to create an opening for the instrument. In at least some embodiments, one or more dilators may be used to create or enlarge the opening.

In at least some embodiments, the spacer 100, in the implantation position, is releasably attached to an implantation tool, such as the spacer insertion instrument 270, as illustrated in FIG. 2A. In at least some embodiments, the spacer 100 is inserted into a port or cannula, if one is employed, which has been operatively positioned to form an opening to the interspinous space within a patient's back. The spacer 100, attached to the spacer insertion instrument 270 or other implantation tool, is inserted into the interspinous space between the spinous processes of two adjacent vertebrae. In at least some embodiments, a clinician can observe with fluoroscopy or other imaging technique the positioning of the spacer 100 inside the patient. The spacer insertion instrument 270 or other implantation tool is operated to rotate the spacer 100 from the implantation position (FIG. 2B) to the deployed position (FIG. 2C) with the first and second arms 104, 106 receiving portions of the two adjacent vertebrae. Following deployment of the spacer 100, the spacer insertion instrument spacer insertion instrument 270 or other implantation tool (and any other instrumentation, such as a cannula or dilator) is removed from the body of the patient leaving the implanted spacer.

U.S. Pat. Nos. 8,123,782; 8,128,662; 8,273,108; 8,277,488; 8,292,922; 8,425,559; 8,613,747; 8,864,828; 8,945,183; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference, illustrate a variety of tools for insertion and deployment of an interspinous spacer between adjacent spinous processes. These tools can be used or modified for insertion and deployment of the interspinous spacers described herein.

FIGS. 2A to 2G illustrate one embodiment of a spacer insertion instrument 270. The spacer insertion instrument 270 includes a cannula 272 connected to a handle 274. As illustrated in FIGS. 2B to 2F, the spacer insertion instrument 270 includes clamps 276 extending distally from the cannula 272 to releasably clamp to the body 102 of the spacer 100. The body 102 of the spacer 100 includes undercut attachment portions 130 (FIG. 1A) on opposite sides of the body 102 for receiving the clamps 276 of the cannula 272, as illustrated in FIGS. 2B to 2F.

In at least some embodiments, the clamps 276 each include a clamp shaft 278 (which may be the same clamp shaft for both clamps or a different clamp shaft for each clamp), a clamp arm 279 extending from the clamp shaft, and a clamp extension 280 extending from a distal portion of the clamp arm, as illustrated in FIGS. 2D and 2E. In at least some embodiments, the clamp shaft 278 can be made of two or more parts or can be single, undifferentiated part. In at least some embodiments, the clamp shaft 278 (or a portion of the clamp shaft), the clamp arm 279, and the clamp extension 280 are a single, undifferentiated part or can be made of two or more parts.

Any suitable method for clamping or unclamping the spacer 100 using the clamps 276 can be used. In at least some embodiments, a mechanism for clamping and unclamping the clamps 276 to the spacer 100 can be controlled using the spacer insertion instrument 270. In at least some embodiments, the spacer insertion instrument 270 includes the same mechanism for clamping and unclamping the spacer 100 as instruments described in the references cited above. In at least some embodiments, the clamp shaft 278 of the clamp 276 has an outwardly slanted portion 277 that fits against a similarly slanted inner shaft 281, as illustrated in FIG. 2D. The clamps 276 can be pushed downwardly relative to the cannula 272 and inner shaft 281 to slide the clamp shaft 278 of the clamp 276 against the inner shaft 281 causing the clamp arms 279 and clamp extensions 280 to spread apart from each other (due to the slanting of the inner shaft 271 and portion 277 of the clamp shaft 278) allowing for the insertion of a spacer 100 between the claim extensions 280. Retracting the clamp shaft 278 relative to the cannula 272 and inner shaft 281 to retract the clamps 276 and move the clamp extensions toward each other to grip the spacer 100.

Any suitable mechanism can be used to move the clamp shaft 278. In at least some embodiments, the handle 274 includes a first portion 273, mechanically coupled to the clamp shaft(s) 278 of the clamps 276, that can be moved downward to spread the clamp extensions 280 for receiving the spacer 100 and then moved upward to grip the spacer.

In at least some embodiments, the clamp extensions 280 fit underneath the undercut attachment portion 130 of the body 102 of the spacer 100, as illustrated in FIGS. 2D and 2E. The clamp extensions 280 of the clamps 276 facilitate gripping the spacer 100 by the spacer insertion instrument 270 during implantation. In at least some embodiments, the clamp extensions 280 include a cylindrical portion for insertion into the undercut attachment portion 130 of the body 102 of the spacer 100, as illustrated in FIG. 2E. In at least some embodiments, the undercut attachment portion 130 extends to the opening 118 and the corresponding cylindrical portion of the clamp extension 280 fits into the opening 118 and allows the corresponding arm 104, 106 to rotate about the clamp extension. In at least some embodiments, as described in more detail below, the arms 104, 106 of the spacer 100 are rotated from the implantation position (FIG. 2B) to the deployed position (FIG. 2C) by rotating around the cylindrical portions of the clamp extensions 280. In at least some embodiments, the cylindrical portion of the clamp extension 280 and the corresponding opening 118 of the corresponding arm 104, 106 have a curvature or radius of curvature that is the same or differs by no more than 5%, 10%, or 25%.

The spacer insertion instrument 270 further includes at least one cam actuator 282 extending from the cannula 272 and a cam shaft 284 attached to the cam actuator(s) and extending along the cannula. In at least some embodiments, the cam shaft 284 can be made of two or more parts or can be single, undifferentiated part. In at least some embodiments, the cam shaft 284 (or a portion of the cam shaft) and the cam actuator(s) 282 are a single, undifferentiated part or can be made of two or more parts.

In the implantation position, the body 102 of the spacer 100 includes cam surfaces 132 that the cam actuator(s) 282 can engage and push against to rotate the arms 104, 106 from the implantation position (FIG. 2B) to the deployed position (FIG. 2C) via intermediate positions, such as the position illustrated in FIG. 2F. In at least some embodiments, the spacer insertion instrument 270 can be operated use the cam shaft 284 to move the cam actuator(s) 282 away from the cannula 272, as seen by comparing FIGS. 2B and 2C. The movement of the cam actuator(s) 282 pushes the cam surfaces 132 and the arms 104, 106 rotate about the clamp extensions 280 from the implantation position to the deployed position. Any mechanism or method for moving the cam shaft can be used. In at least some embodiments, the handle 274 includes a second portion 275 that can be rotated to move the cam shaft 284 and cam actuator(s) 282 downward and, optionally, upward. For example, the second portion 275 of the handle 274 and the proximal end portion of the cam shaft 284 can be threaded and engaged with each other so that the rotation of the second portion of the handle to longitudinal motion of the cam shaft 284 and cam actuator(s) 282. Any other suitable method for moving the first and second arms 104, 106 from the implantation position to the deployed position.

As the first and second arms 104, 106 move to the deployed position (FIG. 2C) the latch 112 extends into the cavity 109a resulting in the deployed arrangement illustrated in FIG. 1A. In at least some instances, it may be useful to reverse the deployment, for example, to explant the interspinous spacer 100. In at least some embodiments, the spacer insertion instrument 270 includes a wedge 286 and wedge shaft 288 to facilitate explantation of the spacer 100. The wedge 286 can be pushed into the cavity 109a to move the latch 112 beyond engagement (or potential engagement) with the lip 114 toward the wall 110a of first arm 104 (see, FIG. 2E). The first and second arms 104, 106 can be rotated back to the implantation position by pulling the spacer 100 away from the vertebra, allowing engagement with an access cannula (for the spacer insertion instrument 270) or the tissue to produce the rotation of the first and second arms.

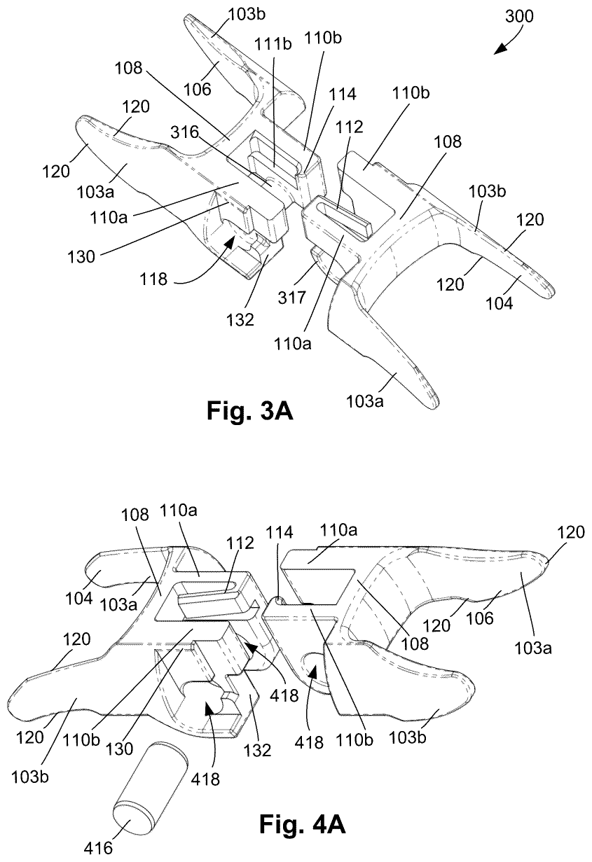

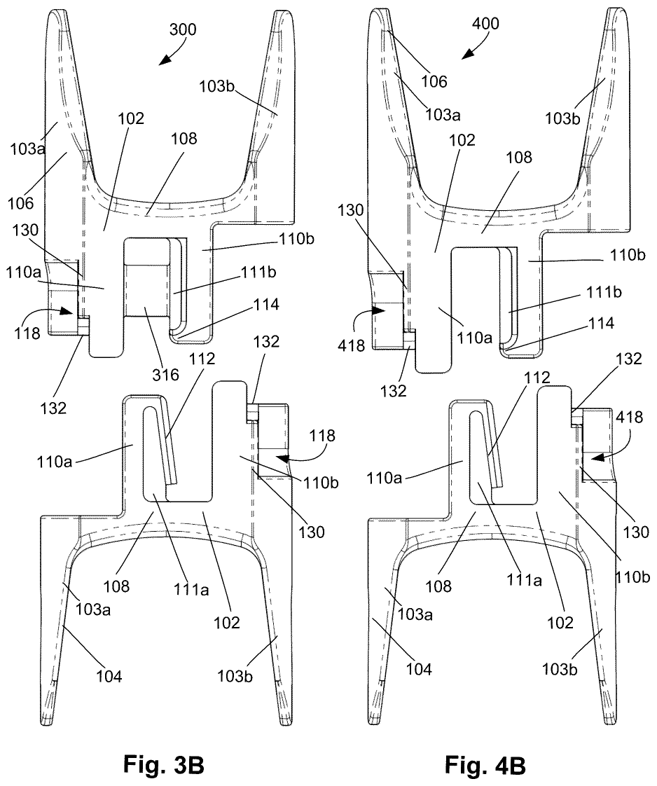

FIGS. 3A and 3B illustrate another embodiment of an interspinous spacer 300. The interspinous spacer 300 is the same and has the same design features and variations and considerations as the interspinous spacer 100, except as described below. The interspinous spacer 300 includes a single post 316 on the second arm 106 (or, alternatively, the first arm 104) and a receiving appendage 317 on the first arm 104 (or, alternatively, the second arm 106) that receives and fits around the single post 316. Interspinous spacer 100 can be modified to include the single post 316 and receiving appendage 317.

In at least some embodiments, the single post 316 extends between at least the two walls 110a, 110b, as illustrated in FIGS. 3A to 3C. In at least some embodiments, the single post 316 and a corresponding inner surface 318 of the receiving appendage 318 have the same curvature or radius of curvature. In at least some embodiments, the use of the post 316 and receiving appendage 317 of the interspinous spacer 300 allows for thicker clamp arms 279 (FIG. 2D) on the clamp 276, which may improve tool stability.

FIGS. 4A and 4B illustrate another embodiment of an interspinous spacer 400. The interspinous spacer 400 is the same and has the same design features and variations and considerations as the interspinous spacers 100, 300, except as described below. The interspinous spacer 400 includes a separate post 416 that fits through openings 418 in the walls 110a, 110b of the first and second arms 104, 106. Interspinous spacer 100 can be modified to include the post 416 and openings 418.

In at least some embodiments, the separate post 416 and the openings 418 have the same curvature or radius of curvature. In at least some embodiments, the use of the post 416 and openings 418 of the interspinous spacer 400 allows for thicker clamp arms 279 (FIG. 2D) on the clamp 276, which may improve tool stability.

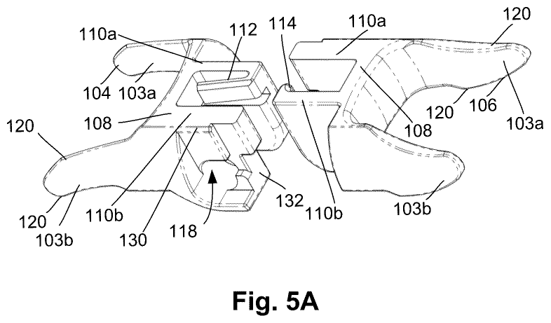

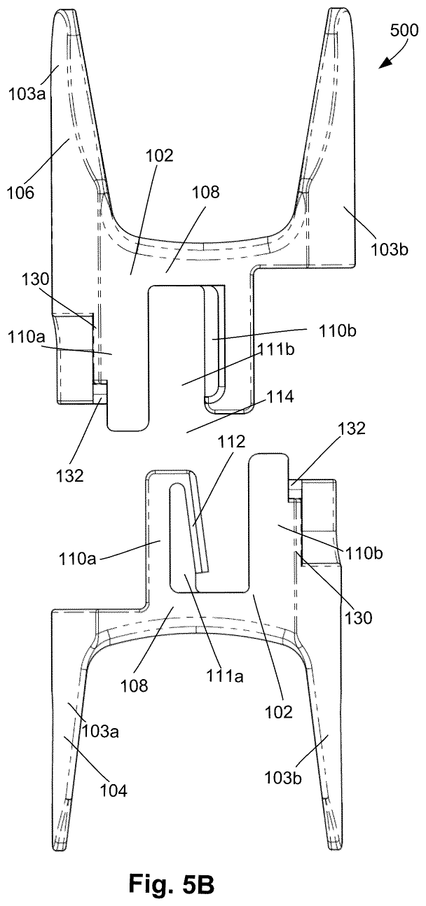

FIGS. 5A and 5B illustrate another embodiment of an interspinous spacer 500. The interspinous spacer 500 is the same and has the same design features and variations and considerations as interspinous spacers 100, 300, 400, except as described below. The interspinous spacer 500 does not include any post 116, 316, 416; openings 118, 418; and receiving appendage 417 so there is no hinge axle for the interspinous spacer 500. The spacer insertion instrument 270 can be used to keep the arms 104, 106 engaged with each other in the implantation position. In the deployed position, the arms 104, 106 are locked by the latch 112 and lip 114.

The above specification provides a description of the manufacture and use of the invention. Since many embodiments of the invention can be made without departing from the spirit and scope of the invention, the invention also resides in the claims hereinafter appended.

What is claimed as new and desired to be protected is:

1. An interspinous spacer, comprising:
a first arm comprising a body and two receiving extensions extending distally from the body, the body comprising a bridge extending between the two receiving extensions, two walls extending proximally from the bridge, and a latch extending distally from a proximal end of a one of the two walls of the body of the first arm and extending between the two walls of the body of the first arm; and
a second arm comprising a body and two receiving extensions extending distally from the body, the body comprising a bridge extending between the two receiving extensions, two walls extending proximally from the bridge, and a lip extending from a proximal end of a one of the two walls of the body of the second arm and extending between the two walls of the body of the second arm;
wherein the first arm and the second arm are coupled together by interleaving the two walls of the first and second arms, wherein the first arm and second arm, when coupled together, are rotatable from an implantation position with the two receiving extensions of both the first arm and second arm extending in a same direction to a deployed position with the two receiving extensions of the first arm extending in an opposite direction from the two receiving extensions of the second arm, wherein, in the deployed position, the latch of the body of the first arm is configured to engage the lip of the body of the second arm when the first arm and the second arm are pulled away from each other to prevent or hinder decoupling of the first arm from the second arm.

2. The interspinous spacer of claim 1, wherein the interspinous spacer consists of the first arm and the second arm.

3. The interspinous spacer of claim 1, wherein each of the first arm and the second arm comprises a post extending from a one of the two walls and an opening in another one of the two walls, wherein the post of each of the first arm and the second arm is configured to extend into the opening of the other one of the first arm and the second arm.

4. The interspinous spacer of claim 1, wherein a one of the first arm or the second arm comprises a receiving appendage extending from the body and another one of the first arm or the second arm comprises a post configured to be received within the receiving appendage.

5. The interspinous spacer of claim 1, further comprising a post, wherein each of the first arm and the second arm comprises an opening in at least one of the two walls for receiving the post extending through each of the openings of the first arm and the second arm.

6. The interspinous spacer of claim 1, wherein a one of the two walls of each of the first arm and the second arm comprises an undercut attachment portion for attachment of a clamp of a spacer insertion instrument.

7. The interspinous spacer of claim 1, wherein the body of each of the first arm and the second arm comprises a cam surface for engagement of a cam actuator of a spacer insertion instrument to facilitate rotation of the first arm and the second arm from the implantation position to the deployed position.

8. The interspinous spacer of claim 1, wherein the latch extends along only a first portion of a depth of the one of the two walls of the body of the first arm.

9. The interspinous spacer of claim 8, wherein, along at least part of a remaining portion of the depth of the one of the two walls of the body of the first arm, the one of the two walls of the body of the first arm extends laterally toward another one of the two walls.

10. The interspinous spacer of claim 8, wherein, along at least part of a remaining portion of the depth of the one of the two walls of the body of the first arm, a portion of a one of the two walls of the body of the second arm extends laterally toward the one of the two walls of the body of the first arm and is configured for positioning adjacent to the latch when the interspinous spacer is in the deployed position.

11. A kit, comprising:
the interspinous spacer of claim 1; and
a spacer insertion instrument configured to releasably grip the interspinous spacer for implantation into a patient.

12. The kit of claim 11, wherein the spacer insertion instrument comprises at least two clamps configured for releasably gripping the interspinous spacer between the clamps and at least one cam actuator configured to engage the first and second arms and, when operated, to rotate the first and second arms from the implantation position to the deployed position.

13. The kit of claim 12, wherein each of the at least two clamps comprises a clamp extension configured for positioning in an opening in the body of one of the first arm or the second arm for the interspinous spacer for releasably gripping the interspinous spacer.

14. The kit of claim 12, wherein the spacer insertion instrument comprises a handle portion that, when operated, spreads the at least two clamps apart for loading or unloading of the interspinous spacer from the spacer insertion instrument.

15. The kit of claim 11, wherein the spacer insertion instrument further comprises a wedge to engage the latch when the interspinous spacer is in the deployed position and to move the latch so that the first and second arms of the interspinous spacer can be rotated from the deployed position toward the implantation position.

16. A method for implanting the interspinous spacer of claim 1, the method comprising:
inserting into a patient the interspinous spacer in the implantation position and coupled to a spacer insertion instrument;
rotating the first and second arms of the interspinous spacer from the implantation position to the deployed position using the spacer insertion instrument;
after the rotating to the deployed position, releasing the interspinous spacer from the spacer insertion instrument; and
removing the spacer insertion instrument from the patient leaving the interspinous spacer implanted in the patient.

17. The method of claim 16, further comprising coupling the interspinous spacer to the spacer insertion instrument using at least two clamps of the spacer insertion instrument.

18. The method of claim 16, wherein the rotating comprises operating a cam actuator of the spacer insertion instrument to push against portions of the first and second arms resulting in the rotation of the first and second arms of the interspinous spacer from the implantation position to the deployed position.

19. The method of claim 16, wherein the rotating comprises disengaging the latch from engagement with the lip allowing the latch to extend into a cavity between defined the by the walls of the first and second arms.

20. A method for explanting the interspinous spacer of claim 1, the method comprising:
coupling a spacer insertion instrument to the interspinous spacer;
operating a wedge of the spacer insertion instrument to engage the latch and move the latch toward the one of the walls of the body of the first arm; and
causing the first arm and the second arm to move from the deployed position toward the implantation position.
